(19) United States
(12) Patent Application Publication
(10) Pub. No.: US 2005/0107466 A1
(43) Pub. Date: May 19, 2005

(54) Method of treating or reducing suicidal thoughts with escitalopram

(75) Inventor: Henning Friis Andersen, Hillerod (DK)

Correspondence Address: DARBY & DARBY P.C., P. O. BOX 5257, NEW YORK, NY, 10150-5257, US

(73) Assignee: H. Lundbeck A/S, Valby-Copenhagen (DK)

(21) Appl. No.: 10/991,954

(22) Filed: Nov. 17, 2004

Related U.S. Application Data
(60) Provisional application No. 60/520,882, filed on Nov. 17, 2003.

(51) Int. Cl. A61K 31/343

(52) U.S. Cl. 514/469

(57) ABSTRACT

The present invention relates to a method of treating a person suffering from suicidal ideation comprising administration of a pharmaceutically effective amount of escitalopram or a pharmaceutically acceptable salt thereof.

This application claims the benefit of U.S. Provisional Application No. 60/520,882 filed Nov. 17, 2003, which is hereby incorporated by reference.

### FIELD OF THE INVENTION

The present invention relates to the use of escitalopram or a pharmaceutically acceptable salt thereof for the treatment or reduction of suicidal thoughts in a person.

### BACKGROUND OF THE INVENTION

Selective serotonin reuptake inhibitors (hereinafter called SSRIs), such as citalopram, have become first-choice therapeutics in the treatment of depression, certain forms of anxiety and social phobias, because they are effective, well-tolerated and have a favourable safety profile compared to the classic tricyclic antidepressants. Escitalopram is the S-enantiomer of citalopram and has the following structure:

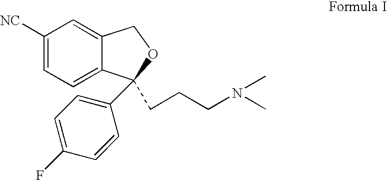

Escitalopram and a method for its preparation is disclosed in U.S. Pat. No. 4,943,590, which is hereby incorporated in its entirety by reference. 5-HT-reuptake inhibition in escitalopram, and accordingly, the potential antidepressant effect of said enantiomer is also disclosed in this patent.

WO 01/03694 A1, which is hereby incorporated in its entirety by reference, relates to the use of escitalopram in the treatment of neurotic disorders, including general anxiety disorder (GAD), social anxiety disorder (SAD), post traumatic stress disorder, and panic attacks, including panic attacks seen in panic disorder, specific phobias, social phobia and angoraphobia.

Anxiety problems are often seen in combination with depression, in the form of either characterized anxiety disorders or associated anxiety symptoms. Such symptoms of anxiety result in an increased risk of suicide and appear to be associated with development of more severe and chronic depressive disorders, Encephale, 28, (5 pt 1) 461-5 (2002 Sep-Oct).

Several investigators have illustrated the importance of depression in suicidal behaviour. More recently, Lönnqvist (2000) has demonstrated that about 60% of suicides involved significant depressive symptomatology and Isacsson G, Lakarttidningen, 2003, 100:2157-2158, has demonstrated a strong association between increasing use of antidepressants and a marked reduction in suicide rates.

Although, suicidal thoughts or behaviour is a part of the spectrum of symptoms in depressive disorders, it should be mentioned that suicidal thoughts or ideation is a necessary, albeit not sufficient prerequisite for suicidal behaviour.

As the risk of suicidal attempts persist until amelioration of the disease, close monitoring of patients during the first weeks of treatment is important.

It has now been found that escitalopram provides early alleviation of suicidal ideation.

### SUMMARY OF THE INVENTION

Accordingly, the present invention relates to the use of escitalopram or a pharmaceutically acceptable salt thereof for the treatment of persons suffering from suicidal thoughts, including persons at risk of committing suicide and persons having recently attempted to commit suicide or having a history of suicide attempts.

In one particular embodiment, the person having suicidal thoughts suffers from depression. In a specific embodiment of the invention, the person suffers from depression and is starting treatment with an antidepressant. In a particular embodiment, the patient is treated with escitalopram for the first 6-8 weeks of treatment with an antidepressant, or at least the first 6-8 weeks of treatment with an antidepressant.

In another specific embodiment of the invention, the person suffering from suicidal thoughts has a score of 2 or above on item 10 (suicidal ideation) on the MADRS depression scale.

According to the invention, the person suffering from depression may suffer from major depression or severe depression, from depression in combination with an anxiety disorder or anxiety symptoms, or the person may suffer from a disorder involving depression symptoms.

According to a specific embodiment of the invention, escitalopram is used as a once daily dosage form containing 10-20 mg of escitalopram or a pharmaceutically acceptable salt thereof calculated as the free base, in particular a once daily dosage form containing 20 mg escitalopram or a pharmaceutically acceptable salt thereof calculated as the free base.

In another particular embodiment of the invention, escitalopram is used in the form of the oxalate salt thereof and in another particular embodiment of the invention, escitalopram is used in the form of the hydrobromide salt thereof.

Another embodiment of the invention is a method of marketing escitalopram or a pharmaceutically acceptable salt thereof (collectively referred to hereafter as “escitalopram”) by marketing, advertising, or selling escitalopram for the treatment or reduction of suicidal thoughts in a person in need thereof. The marketing may be directed to, for example, people suffering from suicidal thoughts and/or doctors treating such people (such as psychologists). The marketing step may comprise the step of including a statement in the labeling for an escitalopram containing pharmaceutical product/composition that the product/composition can treat or reduce suicidal thoughts in a person.

Yet another embodiment is a method of treating a person suffering from suicidal thoughts or ideation comprising the steps of (a) marketing to doctors a pharmaceutical product containing escitalopram (e.g., 10-20 mg escitalopram or a pharmaceutically acceptable salt thereof) as being effective for the treatment of person suffering from suicidal thoughts or ideation; (b) diagnosing a patient as suffering from suicidal thoughts or ideation by a doctor; (c) prescribing the pharmaceutical product to the patient by the doctor in response to the marketing of the pharmaceutical product and the diagnosis of the patient; and (d) administering the prescribed pharmaceutical product to the patient.

Yet another embodiment is a method of treating a person suffering from suicidal thoughts or ideation comprising the steps of (a) providing to the marketplace a pharmaceutical product containing escitalopram (e.g., 10-20 mg escitalopram or a pharmaceutically acceptable salt thereof); (b) sensitizing a doctor to a product claim that the pharmaceutical product is effective for the treatment of person suffering from suicidal thoughts or ideation; (c) diagnosing a patient as suffering from suicidal thoughts or ideation by the doctor; (d) selectively prescribing the pharmaceutical product to the patient by the sensitized doctor in view of the diagnosis of the patient; and (e) orally administering the prescribed pharmaceutical product to the patient.

Yet another embodiment is a method for treating a patient suffering from suicidal thoughts or ideation comprising the steps of (a) purchasing a pharmaceutical product containing escitalopram (e.g., 10-20 mg escitalopram or a pharmaceutically acceptable salt thereof); (b) tendering a prescription for the patient suffering from suicidal thoughts or ideation before completing the purchase transaction, the prescription having been received in response to a diagnosis of suicidal thoughts or ideation by a doctor, the doctor (1) having been sensitized to a product claim that the pharmaceutical product is effective for the treatment of person suffering from suicidal thoughts or ideation, and (2) having selectively prescribed the pharmaceutical product; and (c) administering the purchased pharmaceutical product to the patient.

Yet another embodiment is a therapeutic package comprising (a) a container, (b) a dosage form of escitalopram or a pharmaceutically acceptable salt thereof, and (c) written matter associated with the therapeutic package stating that the dosage form can be administered to treat or reduce suicidal thoughts in a person.

Yet another embodiment is a therapeutic package for dispensing escitalopram or a pharmaceutically acceptable salt thereof to a person being treated for suicidal thoughts. The therapeutic package comprises:

(a) one or more unit doses, each unit dose comprising an effective amount of escitalopram or a pharmaceutically acceptable salt thereof to treat or reduce suicidal thoughts in a person; and

(b) a container containing:
(i) the unit dose or unit doses and
(ii) labeling directing the use of the package, unit dose, or unit doses in the treatment or reduction of suicidal thoughts in a person.

### DETAILED DESCRIPTION OF THE INVENTION

In brief, a clinical trial was designed as a randomised, double-blind, parallel-group trial comprising two different fixed escitalopram doses (10 mg and 20 mg per day) for eight weeks. A placebo-arm was included, as was an active comparator arm in terms of 40 mg citalopram per day.

The persons fulfilled the following criteria: (a) DSM-IV (i) major depression with symptoms present for a minimum of 4 weeks; (b) minimum total score of 22 on the 10-item MADRS (Montgomery SA, Åsberg M., A new depression scale designed to be sensitive to change., Br. J. Psychiatry 1979, 134, 382-389) and a score of 2 or more on the HAM-D item (depressed mood) (Hamilton, M., Br. J. Soc. Clin. Psychol 1967, 6, 278-296). Persons who still fulfilled the MADRS and HAM-D criteria after 1-week, single-blind placebo lead-in were randomised to the double-blind treatment.

Throughout the 8-week double-blind treatment period, persons assigned to placebo or escitalopram 10 mg daily received no adjustment of dosage. Persons in the escitalopram 20 mg daily group and in the citalopram group were titrated to their final dose of after 1 week of treatment at half of their assigned dose. In order to maintain the study blind, all double-blind study medication was administered as 1 capsule per day, regardless of dose or treatment group. No further adjustment of dosage was permitted.

Persons were evaluated at baseline (the end of the 1-week placebo lead-in) and after 1, 2, 4, 6, and 8 weeks. At these clinical visits, the two clinician-rated scales, the Hamilton Depression Rating Scale (HAM-D) and the Montgomery-Åsberg Depression Scale (MADRS) items of suicidal thoughts was used.

It was found that escitalopram at a daily dose of 10-20 mg is more effective in reducing suicidal thoughts than 40 mg citalopram.

Using the results of four clinical studies, it was found that escitalopram is more much more effective for the treatment of the group of patients having a score greater than or equal to 2 on item 10 on the MADRS depression scale compared to citalopram.

Analysis of data from four clinical studies also showed that the overall effect on suicidal thoughts or suicidal ideation are significantly pronounced already after one week of treatment with 10-20 mg escitalopram compared to treatment with placebo.

Finally, it has been found that the percentage of patients experiencing an increase in suicidal thoughts after one week of treatment is significantly lower for the group of patients treated with escitalopram compared to patients treated with citalopram or placebo.

The escitalopram is preferably used as an oxalate salt, preferably a crystalline oxalate salt, or as the hydrobromide salt, preferably a crystalline hydrobromide salt.

Furthermore, in the escitalopram used, R-citalopram is preferably not present in an amount exceeding 2% w/w, most preferred R-citalopram is present in an amount below 1% w/w.

The pharmaceutical composition prepared according to the invention may be used for the treatment of persons suffering from suicidal thoughts or ideation. Some persons having suicidal thoughts suffer from diseases or disorders such as, depression, including major or severe depression, depression with anxiety symptoms in the form of either anxiety disorders as defined in DSM IV (Diagnostic and Statistical manual of Mental Disorders, Fourth Edition, 2000, which is hereby incorporated by reference) or associated anxiety symptoms and in connection with other diseases involving depression.

The phrase “treatment of suicidal thoughts” means a reduction or elimination in suicidal thoughts in a person or patient as measured by the MADRS 10 or HAM-D items of suicidal thoughts, possibly leading to a lower frequency of suicide attempts.

The term “advertising” refers to notifying, informing, and/or apprising one or more individuals of information (e.g., the efficacy of a pharmaceutical product for treating or reducing an indication), such as by mass media, including, but not limited to, newspaper, magazine, and internet advertisements, television commercials, and billboard signs. The term “advertising” as used herein also includes including a statement that the pharmaceutical product can treat or reduce the indication in the labeling for the pharmaceutical product.

The term “marketing” refers to the act or process of selling a product, including, but not limited to, any offer for sale or sale of a product.

The term “pharmaceutical product” refers to any product containing escitalopram or a pharmaceutically acceptable salt thereof, including, but not limited to, escitalopram oxalate, escitalopram hydrobromide, pharmaceutical compositions containing escitalopram or a pharmaceutically acceptable salt thereof, and unit dosage forms, such as tablets and capsules, containing escitalopram or a pharmaceutically acceptable salt thereof.

The pharmaceutical composition prepared according to the invention may comprise escitalopram in a daily dosage form containing 5-20 mg escitalopram, preferably 10-20 mg escitalopram, including 10 mg, 15 mg and 20 mg, and most preferred 20 mg escitalopram (calculated as the free base).

The pharmaceutical composition according to the invention is preferably an oral formulation, preferably a tablet. However, conventional capsule formulations are also a possibility.

Thus, tablets may be prepared by mixing the active ingredient or a granulate thereof with ordinary adjuvants, fillers and/or diluents and subsequently compressing the mixture in a conventional tabletting machine or filling the ingredients in a capsule, for example a gelatine capsule. Examples of adjuvants, fillers or diluents comprise: corn starch, potato starch, talcum, magnesium stearate, gelatine, lactose, gums, and the like. Any other adjuvants or additives usually used for such purposes such as colourings, flavourings, preservatives etc. may be used provided that they are compatible with the active ingredients.

Solutions for injections may be prepared by dissolving the active ingredient and possible additives in a part of the solvent for injection, preferably sterile water, adjusting the solution to desired volume, sterilisation of the solution and filling in suitable ampules or vials. Any suitable additive conventionally used in the art may be added, such as tonicity agents, preservatives, antioxidants, etc.

All references cited herein are incorporated by reference. To the extent that a conflict may exist between the specification and the reference the language of the disclosure made herein controls.

1. A method of treating a person suffering from suicidal thoughts or ideation comprising administering to a person in need thereof an effective amount of escitalopram or a pharmaceutically acceptable salt thereof.

2. The method of claim 1, wherein the person has suicidal thoughts and is at risk of committing suicide.

3. The method of claim 1, wherein the person has recently tried to commit suicide or has a history of suicide attempts.

4. The method of claim 1, wherein the person is suffering from depression.

5. The method of claim 4, wherein the depression is major depression.

6. The method of claim 4, wherein the depression is severe depression.

7. The method of claim 4, wherein the person is further suffering from an anxiety disorder or anxiety symptoms.

8. The method of claim 1, wherein the person has a score of greater than or equal to 2 on item 10 of the MADRS depression scale.

9. The method of claim 1, wherein the person is starting treatment with an antidepressant.

10. The method of claim 9, wherein the effective amount of escitalopram or a pharmaceutically acceptable salt thereof is administered for the first 6-8 weeks of antidepressant treatment.

11. The method of claim 1, wherein the pharmaceutically acceptable salt is the oxalate salt.

12. The method of claim 1, wherein the pharmaceutically acceptable salt is the hydrobromide salt.

13. The method of claim 1, wherein the effective amount is 10-20 mg of escitalopram calculated as the free base.

14. The method of claim 13, wherein the effective amount is 20 mg of escitalopram calculated as the free base.

15. The method of claim 1, wherein the escitalopram or a pharmaceutically acceptable salt thereof is administered once daily.

16. The method of claim 1, wherein the escitalopram or a pharmaceutically acceptable salt thereof is administered orally or via injection.

17. The method of claim 16, wherein the escitalopram or a pharmaceutically acceptable salt thereof is administered in the form of a tablet, capsule, suspension or solution.

18. A therapeutic package comprising
(a) a container,
(b) a dosage form of escitalopram or a pharmaceutically acceptable salt thereof, and
(c) written matter associated with the therapeutic package stating that the dosage form can be administered to treat or reduce suicidal thoughts in a person.

19. A therapeutic package for dispensing escitalopram or a pharmaceutically acceptable salt thereof to a person being treated for suicidal thoughts comprising:
(a) one or more unit doses, each unit dose comprising an effective amount of escitalopram or a pharmaceutically acceptable salt thereof to treat or reduce suicidal thoughts in a person; and
(b) a container containing
(i) the unit dose or unit doses and
(ii) labeling directing the use of the package, unit dose, or unit doses in the treatment or reduction of suicidal thoughts in a person.

20. A method of treating a person suffering from suicidal thoughts or ideation comprising the steps of:
(a) marketing to doctors a pharmaceutical product containing 10-20 mg escitalopram or a pharmaceutically acceptable salt thereof as being effective for the treatment of person suffering from suicidal thoughts or ideation;
(b) diagnosing a patient as suffering from suicidal thoughts or ideation by a doctor;
(c) prescribing the pharmaceutical product to the patient by the doctor in response to the marketing of the pharmaceutical product and the diagnosis of the patient; and
(d) administering the prescribed pharmaceutical product to the patient.

21. A method of treating a person suffering from suicidal thoughts or ideation comprising the steps of:
(a) providing to the marketplace a pharmaceutical product containing 10-20 mg escitalopram or a pharmaceutically acceptable salt thereof;
(b) sensitizing a doctor to a product claim that the pharmaceutical product is effective for the treatment of person suffering from suicidal thoughts or ideation;
(c) diagnosing a patient as suffering from suicidal thoughts or ideation by the doctor;
(d) selectively prescribing the pharmaceutical product to the patient by the sensitized doctor in view of the diagnosis of the patient; and
(e) orally administering the prescribed pharmaceutical product to the patient.

22. A method for treating a patient suffering from suicidal thoughts or ideation comprising the steps of:
(a) purchasing a pharmaceutical product containing 10-20 mg escitalopram or a pharmaceutically acceptable salt thereof;
(b) tendering a prescription for the patient suffering from suicidal thoughts or ideation before completing the purchase transaction, the prescription having been received in response to a diagnosis of suicidal thoughts or ideation by a doctor, the doctor (1) having been sensitized to a product claim that the pharmaceutical product is effective for the treatment of person suffering from suicidal thoughts or ideation, and (2) having selectively prescribed the pharmaceutical product; and
(c) administering the purchased pharmaceutical product to the patient.

23. A method for marketing escitalopram or a pharmaceutically acceptable salt thereof comprising the step of marketing escitalopram or a pharmaceutically acceptable salt thereof for the treatment or reduction of suicidal thoughts in a person.
